(19) United States
(12) Patent Application Publication
(10) Pub. No.: US 2024/0074951 A1
(43) Pub. Date: Mar. 7, 2024

(54) WATER-IN-OIL EMULSIFIED COSMETIC COMPOSITION

(71) Applicant: LVMH RECHERCHE, SAINT-JEAN-DE-BRAYE (FR)

(72) Inventors: Kenta FUJIOKA, Tokyo (JP); Koichi HATA, Tokyo (JP)

(21) Appl. No.: 18/258,896

(22) PCT Filed: Dec. 22, 2020
(86) PCT No.: PCT/EP2020/087631

§ 371 Date: Jun. 22, 2023

(51) Int. Cl. A61K 8/06; A61K 8/891; A61Q 1/02; A61Q 1/12

(52) U.S. Cl. CPC A61K 8/064; A61K 8/891; A61Q 1/02; A61Q 1/12; A61K 2800/30; A61K 2800/43

(57) ABSTRACT

The present invention provides a water-in-oil emulsified cosmetic composition comprising a hydrophobized pigment (A), a silicon film former (B), a silicone-based surfactant having an HLB value of 2 to 8 (C), and optionally a non-volatile oil (D), wherein component (A) is at least one kind of an inorganic pigment except for calcium phosphate, which is treated with a hydrophobizing agent except for a bisbenzoyl compound, the ratio of the mass of component (B) to the total mass of component (C) and component (D) is 1.0 to 9.0, and no fluoroether compound is contained in the composition.

### TECHNICAL FIELD

The present invention relates to a water-in-oil emulsified cosmetic composition.

### BACKGROUND ART

Foundation is used to make the unevenness and pores of the skin inconspicuous and to make the skin beautiful. Various forms such as powder type, liquid type and cream type are known. To maintain a beautiful finish all day long, a long-lasting effect is required for foundation. For example, Japanese Patent Application Laid-Open No. 2005 255623 discloses a water-in-oil type foundation, which claims improved lasting effect.

Currently, the COVID 19 pandemic is occurring around the world, and the wearing of masks is becoming common and essential. In such a situation, non-transfer characteristic, which means that the foundation does not transfer onto the mask, is important in addition to the above-described characteristics. However, since it was rare to wear a mask in a state where makeup was applied, and even if it was worn, it was limited to a short time, the non-transfer characteristic of the foundation was not discussed, and the composition for this purpose is not known until now.

### SUMMARY OF INVENTION

### Technical Problem

Accordingly, it is an object of the present invention to provide a cosmetic composition having not only a good long-lasting effect, a high coverage effect and a comfort finish but also an excellent non-transfer effect to a material (especially a mask) that touches the skin.

### Solution to Problem

The present invention provides a water-in-oil emulsified cosmetic composition comprising a hydrophobized pigment (A), a silicon film former (B), a silicone-based surfactant having an HLB value of 2 to 8 (C), and optionally a non-volatile oil (D), wherein component (A) is at least one kind of an inorganic pigment except for calcium phosphate, which is treated with a hydrophobizing agent except for a bisbenzoyl compound, the ratio of the mass of component (B) to the total mass of component (C) and component (D) is 1.0 to 9.0, and the composition contains no fluoroether compound.

The cosmetic composition of the present invention is a water-in-oil type (W/O type) emulsion cosmetic composition.

Foundations of the oil-in-water type (O/W type), which allows for fresh and comfortable application, are known in the prior art. However, since hydrophilic components (including pigments) are used, it is difficult to increase the adhesion to the skin, and an excellent long-lasting effect cannot be realized. Besides, in the case of conventional W/O type foundations, since it contains a hydrophobic component, a long-lasting effect is superior to that of the O/W type, and a coverage effect can also be improved in comparison to the O/W type by sufficiently dispersing a large amount of the pigment in the oil phase. However, in the conventional W/O type composition, the non-transfer effect to mask cannot be improved because of the stickiness and the thickness of the makeup layer.

The water-in-oil emulsified cosmetic composition of the present invention solves both problems of the above-mentioned O/W type and W/O type, and has not only good long-lasting effect, high coverage effect and comfort finish, but also excellent non-transfer effect to a material (especially a mask) that touches the skin.

First of all, stickiness of component (B) applied to the skin is suppressed mainly because the ratio of the mass of component (B) to the total mass of components (C) and (D) (hereinafter also referred to as “B/(C+D)”) is within a specific range. Further, since a hydrophobized pigment made by a specific hydrophobic agent is used, the makeup layer can be uniform and thin. As a result, the non-transfer effect to mask is greatly improved.

The use of a pigment hydrophobized with a specific hydrophobizing agent makes it possible to increase the amount of the pigment contained in the cosmetic and also makes it possible to improve the dispersibility, thereby improving the high coverage effect. Further, since no remarkable stickiness is produced in the makeup layer and the makeup layer can be formed into a thin film, a comfort finish can be ensured.

When B/(C+D) is less than the lower limit of the range of the present invention, the stickiness becomes excessive, and when B/(C+D) exceeds the upper limit, the function as a foundation is impaired. When a large amount of the conventional pigment is added for the purpose of improving the high coverage effect, the pigment tends to aggregate in component (B), so that a thick and uneven makeup layer is formed. As a result, the non-transfer effect becomes inferior and the comfort finish is impaired.

As described above, in the present invention, all of the non-transfer effect, long-lasting effect, high coverage effect and comfort finish are realized by an exquisite balance between the type of the film-forming agent, the surface treatment of the pigment, and the ratio of the film-forming agent to the non-volatile component (including the surfactant).

As the hydrophobizing agent, at least one compound selected from the group consisting of a silicone compound, a silane compound, an amino acid derivative, a sugar derivative, an organic titanate, a phospholipid, a metal soap, a fatty acid, an oil, and an organic polymer can be used. By using such a hydrophobic agent, the high coverage effect and comfort finish can be further improved while maintaining the non-transfer effect and the long-lasting effect.

As component (B), it is preferable to use at least one component selected from the group consisting of trialkylsiloxy silicate, polyalkylsilsesquioxane, (meth)acrylate silicone, and carbosiloxane dendrimer. By using such components, the non-transfer effect and the long-lasting effect can be further improved while maintaining the high coverage effect and comfort finish.

The water-in-oil emulsion cosmetic composition may further contain at least one compound selected from the group consisting of a polyol, a spherical powder, an organically modified clay mineral and a volatile oil. In the case where the water-in-oil emulsion cosmetic composition contains a polyol, the content thereof is preferably less than 6% by mass based on the total amount. The content of the volatile oil is preferably from 25 to 55% by mass based on the total amount.

The polyol gives moisture retention to the skin and has antiseptic properties. In addition to these properties, by setting the content of the polyol to the above range, the comfort finish can be improved while maintaining the non-transfer effect, long-lasting effect and high coverage effect. The volatile oil gives the cosmetic composition ease of spreading, and by setting the content the volatile oil within the above range, the comfort finish can be further improved while maintaining the non-transfer effect, long-lasting effect, and high coverage effect.

The viscosity of the water-in-oil emulsion cosmetic composition at 25° C. is preferably 1000 mPa·s or less, and preferably the water-in-oil emulsion cosmetic is in the form of a two-phase shaking form. Since the viscosity is low, it is easy to thin the makeup layer, and since the amount of component (C) can be reduced by adopting the two-phase shaking form, it is easy to adjust the value of B/(C+D) to the above range.

The present invention provides a cosmetic process for caring for and/or making-up keratinic materials, comprising the application onto keratinic materials, in particular onto skin, of the water-in-oil emulsified cosmetic composition.

The present invention also provides a cosmetic process, wherein the water-in-oil emulsified cosmetic composition provides to the keratinic materials on which it is applied, non-transfer effect, long-lasting effect, high coverage effect and comfort finish.

### Advantageous Effects of Invention

According to the invention it is possible to provide a cosmetic composition having not only good long-lasting effect, high coverage effect and comfort finish but also excellent non-transfer effect to a material that touches the skin (particularly a mask).

### DESCRIPTION OF EMBODIMENTS

The water-in-oil emulsion cosmetic composition according to the embodiment contains (A) hydrophobized pigment. Component (A) is a hydrophobized pigment in which 1 or 2 or more types of inorganic pigments excluding a calcium phosphate-based pigment are surface-treated with a hydrophobizing agent excluding a bisbenzoyl compound.

Examples of the inorganic pigment to be surface-treated include metallic oxides such as titanium dioxide, iron oxide, zinc oxide, cerium oxide, aluminum oxide, bengala, navy blue, chromium oxide and chromium hydroxides; metallic complexes such as manganese violet and cobalt titanate; and carbon black. Titanium dioxide is known as a white pigment, and iron oxide is known as a red, yellow or black pigment. By appropriately combining these, it is possible to adjust the color to a desired color tone. The inorganic pigment does not include a calcium phosphate pigment such as hydroxyapatite.

The hydrophobizing agent can be at least one compound selected from the group consisting of a silicone compound, a silane compound, an amino acid derivative, a sugar derivative, an organic titanate, a phospholipid, a metal soap, a fatty acid, an oil and an organic polymer. It is preferable to use two or more hydrophobic agents. When two kinds of hydrophobizing agents are used, two kinds among silicone compound, silane compound, amino acid derivative and organic titanate are preferable. The hydrophobizing agent does not include a bisbenzoyl compound such as 4-tert-butyl-4-4-methoxybenzoylmethane.

Examples of the silicone compound include dialkylpolysiloxane such as monomethylpolysiloxane (methicone) and dimethylpolysiloxane (dimethicone). Examples of the silane compound include trialkylalkoxysilanes such as trimethylmethoxysilane and triethoxycaprylylsilane.

The amino acid derivatives can be derivatives of amino acids such as glycine, alanine, sarcosine, proline, hydroxyproline, aspartic acid, glutamic acid, lysine, and the like. Preferred derivatives are acylated amino acids acylated with fatty acids. As the fatty acids used for acylating, saturated or unsaturated fatty acids having 1 to 22 carbon atoms are preferable, and saturated or unsaturated fatty acids having 8 to 20 carbon atoms are more preferable.

Examples of the saturated fatty acids having 8 to 20 carbon atoms include caprylic acid, capric acid, lauric acid, myristic acid, palmitic acid, stearic acid, arachidic acid, behenic acid, lignoceric acid and cerotin acid. Examples of the unsaturated fatty acids having 8 to 20 carbon atoms include myristoleic acid, palmitoleic acid, sapienoic acid, oleic acid, ecaidoic acid, vaccenic acid, linheraideck acid, α-linoleic acid, arachidonic acid, eicosapentaenoic acid, erucic acid and docosahexaenoic acid.

Examples of the acylated amino acids include stearoyl glutamic acid, lauroyl glutamic acid, myristoyl glutamic acid, lauroyl aspartic acid, stearoyl lysine, lauroyl lysine, myristoyl lysine, and the like. Preferred acylated amino acids are stearoyl glutamic acid, myristoyl glutamic acid and lauroyl aspartic acid. The acylated amino acids can be salts with sodium, potassium, calcium, magnesium or aluminum.

Examples of the sugar derivative serving as the hydrophobic agent include polysaccharide fatty acid esters such as dextrin palmitate, dextrin myristate and dextrin isostearate, and glycolipids. Examples of the organic titanate include titanate coupling agents such as isopropyltriisostearoyl titanate. Examples of the phospholipid include lecithin and hydrogenated lecithin, and examples of the fatty acid include lauric acid, myristic acid, stearic acid, isostearic acid and palmitic acid. Fatty acid esters and fatty acid amides are included to the fatty acids and these also can be used.

Examples of the metal soap serving as the hydrophobic agent include stearic acid soap, 12-hydroxystearic acid soap, behenic acid soap, montanic acid soap, and lauric acid soap. Examples of the oil include wax and fatty oil. Examples of the organic polymer include a urethane compound, an acrylic compound, a (meth)acrylic compound, polyisobutylene and polyethylene.

In addition to the above hydrophobizing agent, aluminum hydroxide can be used as a surface treating agent in obtaining the hydrophobized pigment. In particular, for the inorganic pigment having a catalytic activity such as titanium oxide, aluminum hydroxide is effective for blocking the catalytic activity.

Component (A) is preferably from 5 to 35% by mass, more preferably from 10 to 30% by mass, and still more preferably from 15 to 25% by mass, based on the total amount of the cosmetic composition. When the content of component (A) is within the above range, the high coverage effect and the long-lasting effect are further enhanced, and a thinner makeup film can be formed.

With respect to component (A), the mass of the portion derived from the hydrophobic agent is preferably from 1 to 15 mass %, more preferably from 2 to 12 mass %, and even more preferably from 3 to 10 mass %, based on the total mass of the hydrophobized pigment. When the content of the hydrophobizing agent is within the above range, the dispersibility of the pigment is further improved, and the high coverage effect and the long-lasting effect are further enhanced. Furthermore, the oil absorbability of the pigment is suppressed, the viscosity of the cosmetic composition is lowered, and a thinner makeup film can be formed.

The water-in-oil emulsion cosmetic composition according to the embodiment contains (B) a film-forming agent containing silicon atom. Compound (B) is also termed silicon film former. As component (B), trialkylsiloxy silicate, polyalkylsilsesquioxane, (meth)acrylate silicone, and carbosiloxane dendrimer can be employed. As the alkyl groups in the trialkylsiloxysilicates and polyalkylsilsesquioxanes, alkyl groups having 1 to 3 carbon atom numbers are preferable, and methyl group, ethyl group, 1-propyl group, and 2-propyl group (isopropyl group) are included. Further, trimethylsiloxy silicate is preferable as the trialkylsiloxy silicate, and polymethylsilsesquioxane is preferable as the polyalkylsilsesquioxane. Among these, trialkylsiloxy silicate, particularly trimethylsiloxy silicate, is preferred.

Component (B) can be handled in a state of being dissolved in a volatile oil. In this case, component (B) can be used together with the volatile oil. Such volatile oils include cyclopentasiloxane, metyltrimethicone, dimethicone, isododecane, butyl acetate and the like. When the amount of the volatile oil is calculated as described later, the amount of the volatile oil used as a solvent of component (B) should be added.

The content of component (B) can be from 2 to 12 mass %, preferably from 3 to 10 mass %, and more preferably from 4 to 8 mass %, based on the total amount of the cosmetic composition. When the content of component (B) is in the above-described range, not only the high coverage effect and the long-lasting effect are further improved, but also the cosmetic can be more excellent in comfort finish.

The water-in-oil emulsified cosmetic composition according to the embodiment contains (C) a silicone-based surfactant having an HLB value of 2 to 8.

Component (C) has a chemical structure comprising a hydrophilic group is introduced into a polysiloxane skeleton, or into a side chain. The HLB value of the silicone surfactant varies with the type of hydrophilic group. Examples of the hydrophilic group include polyether group such as polyethylene glycol (hereinafter also referred to as “PEG”) and polypropylene glycol (hereinafter also referred to as “PPG”), and polyglycerol group. Component (C) can be further modified with an alkyl group (such as polyether-alkyl comodification, and polyglycerol-alkyl comodification). The silicone chain of component (C) can be linear or branched. Two or more silicone chains can be crosslinked by hydrophilic groups. The number of ethylene oxide units in the polyethylene glycol is not particularly limited, and can be 1 or more, 2 or more, 3 or more, 4 or more, 5 or more, or 6 or more. The number of ethylene oxide units can be 11 or less, 10 or less, or 9 or less.

The HLB value of component (C) can be 2 to 8, 2 to 7, 3 to 7, 3 to 6 or 4 to 6. The HLB value of component (C) may also be 1 or higher, 2 or higher or 3 or higher. Examples of component (C) include the commercial products KF-6012® (PEG/PPG-20/22 butyl ether dimethicone, HLB=7.0), KF-60150 (PEG-3 dimethicone, HLB=4.5), KF-6016® (PEG-9 methyl ether dimethicone, HLB=4.5), KF-6017® (PEG-10 dimethicone, HLB=4.5), KF-6028® (PEG-9 polymethylsiloxyethyl dimethicone, HLB=4.0), KF-6038® (lauryl PEG-9 polymethylsiloxyethyl dimethicone, HLB=3.0), KF-6048® (cetyl PEG/PPG-10/1 dimethicone, HLB=3.5), KF-6100®, KF-6104®, KE-6105® and KF-6106® (trade names of Shin-Etsu Chemical Co., Ltd.), and mixtures thereof.

The content of component (C) is preferably from 1 to 5 mass % based on the total amount of the cosmetic composition. When the content of the component (C) is within the above range, the emulsified state is more stabilized, and the high coverage effect and the long-lasting effect can be further enhanced.

The water-in-oil emulsified cosmetic composition according to the embodiment may contain (D) a non-volatile oil. Examples of component (D) include cyclic alkylene carbonate (e.g. propylene carbonate), fatty alcohol of 8 to 22 carbon atoms (e.g. octyldodecanol), fatty ester of mono- or polyhydric alcohol (e.g. isopropyl myristate, isononyl isononanoate, pentaerythrityl tetraisostearate, castor oil), alkyl benzoate, aliphatic hydrocarbon (e.g. hydrogenated polyisobutene), liquid lanolin, olive oil, mineral oil, squalane, polysiloxanes (e.g. dimethicone), aromatic polysiloxanes (e.g. phenyltrimethicone). The term “non-volatile oil” refers to a non-vaporizing oil component that is liquid under normal temperature (i.e. in the range from 15 to 25° C.) and normal pressure (i.e. 1 atmospheric pressure).

The content of component (D) is preferably 8% by mass or less, more preferably 6% by mass or less, and still more preferably 4% by mass or less, based on the total amount of the cosmetic composition. The content can be 0 mass %. When the content of component (D) is within the above range, it is unlikely to produce a dryness-induced stiff feeling or a skin suffocating feeling after application onto skin, and it could produce comfort finish.

The water-in-oil emulsified cosmetic composition according to the embodiment may contain (E1) a volatile oil. Component (E1) is an oily component which is liquid under normal temperature and normal pressure and gradually vaporizes. Examples of component (E1) include cyclopentasiloxane, metyltrimethicone, octamethyltrisiloxane, dimethicone, butyl acetate, isododecane, isohexadecane and a petroleum volatile.

The content of component (E1) can be from 25 to 55 mass %, preferably from 30 to 50 mass %, and more preferably from 35 to 45 mass %, based on the total amount of the cosmetic composition. When the content of component (E1) is in the above range, it is possible to form a thinner coated film without impairing the coverage effect and lasting effect, and the comfort finish is also superior. In addition, it is unlikely to produce a dryness-induced stiff feeling, skin irritation or a skin suffocating feeling after application onto skin, which is more preferred from the viewpoint of the feel during use.

The water-in-oil emulsified cosmetic composition according to the embodiment may contain (E2) an organically modified clay mineral. Component (E2) is a clay modified to be hydrophobic with an organic molecule, and has a thickening effect.

Examples of the clay used for composition (E2) include kaolinite, dickite, nacrite, donbassite, antigorite, berthierine, pyrophyllite, montmorillonite, beidellite, vermiculite, talc, stevensite, hectorite, saponite, chlorite and sepiolite, which are modified to hydrophobicity with an organic molecule. Examples of an organic molecule include quaternary ammonium salts, and more specifically disteardimonium chloride and stearalkonium chloride. Component (E2) includes disteardimonium hectorite and stearalkonium hectorite.

Component (E2) can be handled in a state dispersed in a volatile oil and/or a non-volatile oil, in which case it can be utilized together with a volatile oil.

Such volatile oils include octamethyltrisiloxane, isododecane, isohexadecane, petroleum volatiles and methyltrimethicone, ethanol, in particular isododecane, methyltrimethicone and mixtures thereof. Non-volatile oils include propylene carbonate, castor oil, olive fruit oil, octyldodecanol, isopropyl myristate, liquid lanolin, mineral oil, hydrogenated polyisobutene, phenyltrimethicone, pentaerythrityl tetraisostearate and alkyl benzoate. The amount of the volatile oil or the non-volatile oil coexisting with component (E2) is added as the amount of the volatile oil or the non-volatile oil described below. Component (E2) includes the BENTON GEL® Series, such as BENTON GEL® ISD V (trade name of Elementis).

By containing component (E2), stability of the emulsified state is improved, water resistance and sebum resistance after application to the skin are excellent, and bleeding due to sebum or the like can be suppressed. The content of component (E2) can be from 0.05 to 1.5 mass %, preferably from 0.1 to 1.2 mass %, and more preferably from 0.5 to 1.0 mass %, based on the total amount of the cosmetic composition.

The water-in-oil emulsified cosmetic composition according to the embodiment may contain (E3) a dispersing agent. Component (E3) includes polyhydroxy stearic acid. The content of component (E3) is preferably from 0.05 to 2.0% by mass, and more preferably from 0.1 to 1.0% by mass, based on the total amount of the cosmetic composition.

The water-in-oil emulsion cosmetic composition according to the embodiment contains (F) an aqueous phase. Component (F) contains (F1) water, and may contain at least one of (F2) polyol and (F3) monool.

The content of component (F1) can be from 3 to 25 mass %, preferably from 5 to 22 mass %, and more preferably from 7 to 20 mass %, based on the total mass of the cosmetic composition. When the content of component (F1) is within the above range, the emulsified state is less likely to be unstable, the viscosity of the cosmetic composition is further reduced, and a thin makeup film is easily formed.

Examples of component (F2) include a polyol having 1 to 8 carbon atoms. Examples of component (F2) include ethylene glycol, 1,2-propylene glycol, 1,3-propylene glycol, glycerin, 1,3-butylene glycol, 1,2-pentanediol, 1,2-hexanediol, and 1,2-octanediol. The content of component (F2) can be less than 6% by mass, preferably less than 4% by mass, and more preferably less than 3% by mass, based on the total amount of the cosmetic composition. When the content of component (F2) is within the above range, the moisture retaining effect can be further enhanced without destabilizing the emulsified state.

Examples of compound (F3) include a monool having 1 to 3 carbon atoms. Examples of component (F3) include ethanol, 1-propanol and 2-propanol (isopropanol). The content of component (F3) can be from 1 to 25 mass %, and preferably from 5 to 20 mass %, based on the total amount of the cosmetic composition. When the content of component (F3) is within the above range, a uniform film can be easily formed, and the high coverage effect and the long-lasting effect can be further enhanced.

The water-in-oil emulsified cosmetic composition according to the embodiment can contain (G) spherical powder. Component (G) includes silica, cellulose, starch, and mixtures thereof, and hydrophobized silica, hydrophobized cellulose and hydrophobized starch are preferred, and hydrophobized silica is more preferred. The content of component (G) may be from 0.1 to 10% by mass, and preferably from 0.5 to 5% by mass, based on the total amount of the cosmetic composition.

The water-in-oil emulsified cosmetic composition according to the embodiment can further contain an antibacterial agent, an antioxidant, a perfume, an active ingredient, etc.

In the water-in-oil emulsion cosmetic composition according to the embodiment, the ratio of the mass of component (B) to the total mass of component (C) and component (D) is 1.0 to 9.0. This ratio is preferably 1.2 to 9.0, more preferably 1.5 to 9.0, and still more preferably 2.0 to 9.0. When the components other than component (D) contain non-volatile oil, the above ratio is obtained by including them. In addition, even when the component (E1) (volatile oil) is included, this content is not used in the calculation of the above ratio.

The viscosity of the water-in-oil emulsion cosmetic composition according to the embodiment at 25° C. is preferably 1000 mPas or less, and more preferably 800 mPas or less. The viscosity is measured by using a rotary viscometer (for example, Rheolab QC manufactured by Anton Parr Co., Ltd.), and ST22-4V-40 type spindle is inserted into a water-in-oil emulsified cosmetic composition held at 25° C. in a vessel having an inner diameter of 4.5 cm, and the rotation speed is measured at 100 rpm for 3 minutes. The form of the water-in-oil emulsion cosmetic composition is preferably a two-phase shaking form because the content of component (C) can be reduced.

The water-in-oil emulsion cosmetic composition according to the embodiment does not contain a fluoroether compound such as C₄F₉OCH₃, C₄F₉OC₂H₅, C₅Fii₀C₂H₅, C₃F₇OC₄H₉ and C₄F₉OC₄H₉.

The water-in-oil emulsion cosmetic composition according to the embodiment can be produced, for example, by the following steps. Specifically, components (A), (B), (C), (D), (E1), (E2), (E3) and (G) are mixed to obtain a mixture. Separately, components (F1), (F2) and (F3) are mixed to obtain a mixture. The water-in-oil emulsion cosmetic composition can be obtained by finally mixing the two mixtures.

### EXAMPLES

The present invention will now be explained by the following examples, with the understanding that the invention is not limited by the examples.

### Examples 1 to 19 and Comparative Examples 1 to 6

Water-in-oil emulsified cosmetic compositions were prepared according to the following steps 1 to 3 with the compositions shown in Tables 1 to 3.
Step 1: (A) a pigment (hydrophobized pigment or non-hydrophobized pigment), (B) a silicon film former, (C) a silicone-based surfactant having a low HLB value (HLB value: 2 to 8), (D) a non-volatile oil, (E1) a volatile oil, (E2) an organically modified clay mineral, (E3) a dispersing agent, and (G) a spherical powder were mixed at room temperature using a homogenizer.
Step 2: (F) The aqueous phase was mixed using a homogenizer at room temperature.
Step 3: The mixture obtained in Step 2 was added to the mixture obtained in Step 1 and emulsified using a homogenizer.

The notation of the compounds in Tables 1 to 3 is as follows.
Hydrophobized pigment 1: Titanium Dioxide, Aluminum Hydroxide (92%), Dimethicone, Disodium Stearoyl Glutamate (8%)
Hydrophobized pigment 2: Iron Oxide (YELLOW), Aluminum Hydroxide (92%), Dimethicone, Disodium Stearoyl Glutamate (8%)
Hydrophobized pigment 3: Iron Oxide (RED), Aluminum Hydroxide (92%), Dimethicone, Disodium Stearoyl Glutamate (8%)
Hydrophobized pigment 4: Iron Oxide (BLACK), Aluminum Hydroxide (92%), Dimethicone, Disodium Stearoyl Glutamate (8%)
Hydrophobized pigment 5: Titanium Dioxide (98%), Dimethicone (2%)
Hydrophobized pigment 6: Iron Oxide (YELLOW) (98%), Dimethicone (2%)
Hydrophobized pigment 7: Iron Oxide (RED) (98%), Dimethicone (2%)
Hydrophobized pigment 8: Iron Oxide (BLACK) (98%), Dimethicone (2%)
Hydrophobized pigment 9: Titanium Dioxide, Aluminum Hydroxide (97%), Sodium Myristoyl Glutamate (3%)
Hydrophobized pigment 10: Iron Oxide (YELLOW), Aluminum Hydroxide (97%), Sodium Myristoyl Glutamate (3%)
Hydrophobized pigment 11: Iron Oxide (RED), Aluminum Hydroxide (97%), Sodium Myristoyl Glutamate (3%)
Hydrophobized pigment 12: Iron Oxide (BLACK), Aluminum Hydroxide (97%), Sodium Myristoyl Glutamate (3%)
Hydrophobized pigment 13: Titanium Dioxide, Aluminum Hydroxide (98%), Triethoxycaprylylsilane (2%)
Hydrophobized pigment 14: Iron Oxide (YELLOW) (98%), Triethoxycaprylylsilane (2%)
Hydrophobized pigment 15: Iron Oxide (RED) (98%), Triethoxycaprylylsilane (2%)
Hydrophobized pigment 16: Iron Oxide (BLACK) (98%), Triethoxycaprylylsilane (2%)
Hydrophobized pigment 17: Titanium Dioxide, Aluminum Hydroxide (98%), Isopropyl Titanium Triisostearate, Sodium Lauroyl Aspartate (2%)
Hydrophobized pigment 18: Iron Oxide (YELLOW) (98%), Isopropyl Titanium Triisostearate, Sodium Lauroyl Aspartate (2%)
Hydrophobized pigment 19: Iron Oxide (RED) (98%), Isopropyl Titanium Triisostearate, Sodium Lauroyl Aspartate (2%)
Hydrophobized pigment 20: Iron Oxide (BLACK) (98%), Isopropyl Titanium Triisostearate, Sodium Lauroyl Aspartate (2%)

TABLE 1

| | | Exam- | Exam- | Exam- | Exam- | Exam- | Exam- | Exam- | Exam- | Exam- |
|---|---|---|---|---|---|---|---|---|---|---|
| | | ple1 | ple2 | ple3 | ple4 | ple5 | ple6 | ple 7 | ple8 | ple9 |
| Component (A) | Hydrophobized pigment 1 | 17.3 | 17.3 | 17.3 | 17.3 | 17.3 | 17.3 | 17.3 | 17.3 | 17.3 |
| (Pigment) | Hydrophobized pigment 2 | 2 | 2 | 2 | 2 | 2 | 2 | 2 | 2 | 2 |
| | Hydrophobized pigment 3 | 0.5 | 0.5 | 0.5 | 0.5 | 0.5 | 0.5 | 0.5 | 0.5 | 0.5 |
| | Hydrophobized pigment 4 | 0.2 | 0.2 | 0.2 | 0.2 | 0.2 | 0.2 | 0.2 | 0.2 | 0.2 |
| | Hydrophobized pigment 5 | — | — | — | — | — | — | — | — | — |
| | Hydrophobized pigment 6 | — | — | — | — | — | — | — | — | — |
| | Hydrophobized pigment 7 | — | — | — | — | — | — | — | — | — |
| | Hydrophobized pigment 8 | — | — | — | — | — | — | — | — | — |
| | Hydrophobized pigment 9 | — | — | — | — | — | — | — | — | — |
| | Hydrophobized pigment 10 | — | — | — | — | — | — | — | — | — |
| | Hydrophobized pigment 11 | — | — | — | — | — | — | — | — | — |
| | Hydrophobized pigment 12 | — | — | — | — | — | — | — | — | — |
| | Hydrophobized pigment 13 | — | — | — | — | — | — | — | — | — |
| | Hydrophobized pigment 14 | — | — | — | — | — | — | — | — | — |
| | Hydrophobized pigment 15 | — | — | — | — | — | — | — | — | — |
| | Hydrophobized pigment 16 | — | — | — | — | — | — | — | — | — |
| | Hydrophobized pigment 17 | — | — | — | — | — | — | — | — | — |
| | Hydrophobized pigment 18 | — | — | — | — | — | — | — | — | — |
| | Hydrophobized pigment 19 | — | — | — | — | — | — | — | — | — |
| | Hydrophobized pigment 20 | — | — | — | — | — | — | — | — | — |
| Component (B) | Trimethylsiloxysilicate | 6 | 2.5 | 3 | 4 | 8 | 12 | 6 | 6 | 8 |
| (Film former) | Trimethylsiloxysilicate(60%), | — | — | — | — | — | — | — | — | — |
| | Methyl Trimethicone(40%) | | | | | | | | | |
| | Polymethylsilsesquioxane | — | — | — | — | — | — | — | — | — |
| | Acrylates/Dimethicone | — | — | — | — | — | — | — | — | — |
| | Copolymer(40%), | | | | | | | | | |
| | Methyl Trimethicone(60%) | | | | | | | | | |
| Component (C) | PEG-9 | 2 | 2 | 2 | 2 | 2 | 2 | 1.5 | 4.5 | 1.5 |
| (Low HLB silicone | Polydimethylsiloxyethyl | | | | | | | | | |
| surfactant) | Dimethicone | | | | | | | | | |
| Component (D) | Phenyl Trimethicone | 0 | 0 | 0 | 0 | 0 | 0 | 0 | 0 | 4.5 |
| (Non-volatile | | | | | | | | | | |
| oils) | | | | | | | | | | |
| Component (E) | | | | | | | | | | |
| E1 (Volatile oil) | Methyl Trimethicone | 36 | 39.5 | 39 | 38 | 34 | 30 | 36.5 | 33.5 | 30 |
| E2 (Organically | Disteardimonium | 8 | 8 | 8 | 8 | 8 | 8 | 8 | 8 | 8 |
| modified clay | Hectorite(10%), | | | | | | | | | |
| mineral) | Isododecane(87%) | | | | | | | | | |
| | Propylene Carbonate(3%) | | | | | | | | | |
| E3 (Dispersing | Polyhydroxystearic Acid | 0.5 | 0.5 | 0.5 | 0.5 | 0.5 | 0.5 | 0.5 | 0.5 | 0.5 |
| agent) | | | | | | | | | | |
| Component (F) | | | | | | | | | | |
| (Aqueous phase) | | | | | | | | | | |
| F1 (Water) | Water | 12.5 | 12.5 | 12.5 | 12.5 | 12.5 | 12.5 | 12.5 | 12.5 | 12.5 |
| F2 (Polyol) | Glycerin | 2 | 2 | 2 | 2 | 2 | 2 | 2 | 2 | 2 |
| F3 (Monol) | Ethanol | 12 | 12 | 12 | 12 | 12 | 12 | 12 | 12 | 12 |
| Component (G) | Silica, Dimethicone | 1 | 1 | 1 | 1 | 1 | 1 | 1 | 1 | 1 |
| (Spherical | | | | | | | | | | |
| powder) | | | | | | | | | | |
| SUM | | 100 | 100 | 100 | 100 | 100 | 100 | 100 | 100 | 100 |

TABLE 2

| | | Exam- | Exam- | Exam- | Exam- | Exam- | Exam- | Exam- | Exam- | Exam- | Exam- |
|---|---|---|---|---|---|---|---|---|---|---|---|
| | | ple 10 | ple 11 | ple 12 | ple 13 | ple 14 | ple 15 | ple 16 | ple 17 | ple 18 | ple 19 |
| Component (A) | Hydrophobized pigment 1 | 17.3 | 17.3 | 17.3 | — | — | — | — | 17.3 | 17.3 | 17.3 |
| (Pigment) | Hydrophobized pigment 2 | 2 | 2 | 2 | — | — | — | — | 2 | 2 | 2 |
| | Hydrophobized pigment 3 | 0.5 | 0.5 | 0.5 | — | — | — | — | 0.5 | 0.5 | 0.5 |
| | Hydrophobized pigment 4 | 0.2 | 0.2 | 0.2 | — | — | — | — | 0.2 | 0.2 | 0.2 |
| | Hydrophobized pigment 5 | — | — | — | 17.3 | — | — | — | — | — | — |
| | Hydrophobized pigment 6 | — | — | — | 2 | — | — | — | — | — | — |
| | Hydrophobized pigment 7 | — | — | — | 0.5 | — | — | — | — | — | — |
| | Hydrophobized pigment 8 | — | — | — | 0.2 | — | — | — | — | — | — |
| | Hydrophobized pigment 9 | — | — | — | — | 17.3 | — | — | — | — | — |
| | Hydrophobized pigment 10 | — | — | — | — | 2 | — | — | — | — | — |
| | Hydrophobized pigment 11 | — | — | — | — | 0.5 | — | — | — | — | — |
| | Hydrophobized pigment 12 | — | — | — | — | 0.2 | — | — | — | — | — |
| | Hydrophobized pigment 13 | — | — | — | — | — | 17.3 | — | — | — | — |
| | Hydrophobized pigment 14 | — | — | — | — | — | 2 | — | — | — | — |
| | Hydrophobized pigment 15 | — | — | — | — | — | 0.5 | — | — | — | — |
| | Hydrophobized pigment 16 | — | — | — | — | — | 0.2 | — | — | — | — |
| | Hydrophobized pigment 17 | — | — | — | — | — | — | 17.3 | — | — | — |
| | Hydrophobized pigment 18 | — | — | — | — | — | — | 2 | — | — | — |
| | Hydrophobized pigment 19 | — | — | — | — | — | — | 0.5 | — | — | — |
| Component (B) | Trimethylsiloxysilicate | 8 | 9 | 11 | 6 | 6 | 6 | 6 | — | — | — |
| (Film former) | Trimethylsiloxysilicate(60%), | — | — | — | — | — | — | — | 10 | — | — |
| | Methyl Trimethicone(40%) | | | | | | | | | | |
| | Polymethylsilsesquioxane | — | — | — | — | — | — | — | — | 6 | — |
| | Acrylates/Dimethicone | — | — | — | — | — | — | — | — | — | 15 |
| | Copolymer(40%), | | | | | | | | | | |
| | Methyl Trimethicone(60%) | | | | | | | | | | |
| Component (C) | PEG-9 | 1.5 | 1 | 1 | 2 | 2 | 2 | 2 | 2 | 2 | 2 |
| (Low HLB | Polydimethylsiloxyethyl | | | | | | | | | | |
| silicone | Dimethicone | | | | | | | | | | |
| surfactant) | | | | | | | | | | | |
| Component (D) | Phenyl Trimethicone | 6.5 | 0 | 0 | 0 | 0 | 0 | 0 | 0 | 0 | 0 |
| (Non-volatile | | | | | | | | | | | |
| oils) | | | | | | | | | | | |
| Component (E) | | | | | | | | | | | |
| E1 (Volatile | Methyl Trimethicone | 28 | 34 | 32 | 36 | 36 | 36 | 36 | 32 | 36 | 27 |
| oil) | | | | | | | | | | | |
| E2 (Organically | Disteardimonium | 8 | 8 | 8 | 8 | 8 | 8 | 8 | 8 | 8 | 8 |
| modified clay | Hectorite(10%), | | | | | | | | | | |
| mineral) | Isododecane(87%) | | | | | | | | | | |
| | Propylene Carbonate(3%) | | | | | | | | | | |
| E3 (Dispersing | Polyhydroxystearic Acid | 0.5 | 0.5 | 0.5 | 0.5 | 0.5 | 0.5 | 0.5 | 0.5 | 0.5 | 0.5 |
| agent) | | | | | | | | | | | |
| Component (F) | | | | | | | | | | | |
| (Aqueous phase) | | | | | | | | | | | |
| F1 (Water) | Water | 12.5 | 12.5 | 12.5 | 12.5 | 12.5 | 12.5 | 12.5 | 12.5 | 12.5 | 12.5 |
| F2 (Polyol) | Glycerin | 2 | 2 | 2 | 2 | 2 | 2 | 2 | 2 | 2 | 2 |
| F3 (Monol) | Ethanol | 12 | 12 | 12 | 12 | 12 | 12 | 12 | 12 | 12 | 12 |
| Component (G) | Silica, Dimethicone | 1 | 1 | 1 | 1 | 1 | 1 | 1 | 1 | 1 | 1 |
| (Spherical | | | | | | | | | | | |
| powder) | | | | | | | | | | | |
| SUM | | 100 | 100 | 100 | 100 | 100 | 100 | 100 | 100 | 100 | 100 |

TABLE 3

| | | Comp. | Comp. | Comp. | Comp. | Comp. | Comp. |
|---|---|---|---|---|---|---|---|
| | | Example | Example | Example | Example | Example | Example |
| | | 1 | 2 | 3 | 4 | 5 | 6 |
| Component (A) | Hydrophobized pigment 1 | 17.3 | 17.3 | 17.3 | 17.3 | 17.3 | — |
| (Pigment) | Hydrophobized pigment 2 | 2 | 2 | 2 | 2 | 2 | — |
| | Hydrophobized pigment 3 | 0.5 | 0.5 | 0.5 | 0.5 | 0.5 | — |
| | Hydrophobized pigment 4 | 0.2 | 0.2 | 0.2 | 0.2 | 0.2 | — |
| | Titanium Dioxide | — | — | — | — | — | 17.3 |
| | Iron Oxide(YELLOW) | — | — | — | — | — | 2 |
| | Iron Oxide(RED) | — | — | — | — | — | 0.5 |
| | Iron Oxide(BLACK) | — | — | — | — | — | 0.2 |
| Component (B) | Trimethylsiloxysilicate | 0 | 6 | 6 | 7 | 12 | 6 |
| (Film former) | | | | | | | |
| Component (C) | PEG-9 | 2 | 8 | 2 | 3 | 1 | 2 |
| (Low HLB silicone | Polydimethylsiloxyethyl | | | | | | |
| surfactant) | Dimethicone | | | | | | |
| Component (D) | Phenyl Trimethicone | 0 | 0 | 10 | 4.5 | 0 | 0 |
| (Non-volatile | | | | | | | |
| oils) | | | | | | | |
| Component (E) | | | | | | | |
| E1 (Volatile oil) | Methyl Trimethicone | 42 | 30 | 26 | 34 | 31 | 36 |
| E2 (Organically | Disteardimonium | 8 | 8 | 8 | 8 | 8 | 8 |
| modified clay | Hectorite(10%), | | | | | | |
| mineral) | Isododecane(87%) | | | | | | |
| | Propylene Carbonate(3%) | | | | | | |
| E3 (Dispersing | Polyhydroxystearic Acid | 0.5 | 0.5 | 0.5 | 0.5 | 0.5 | 0.5 |
| agent) | | | | | | | |
| Component (F) | | | | | | | |
| (Aqueous phase) | | | | | | | |
| F1 (Water) | Water | 12.5 | 12.5 | 12.5 | 12.5 | 12.5 | 12.5 |
| F2 (Polyol) | Glycerin | 2 | 2 | 2 | 2 | 2 | 2 |
| F3 (Monol) | Ethanol | 12 | 12 | 12 | 12 | 12 | 12 |
| Component (G) | Silica, Dimethicone | 1 | 1 | 1 | 1 | 1 | 1 |
| (Spherical | | | | | | | |
| powder) | | | | | | | |
| SUM | | 100 | 100 | 100 | 104.5 | 100 | 100 |

The properties of the water-in-oil emulsion cosmetic compositions of Examples and Comparative Examples were measured according to the following method.

[Evaluation of Non-Transfer to Mask, Long-Lasting, Coverage and Comfort Finish]

When the water-in-oil type emulsified cosmetic compositions of Examples and Comparative Examples were applied to the skin of 10 persons (25 to 55 years of age) of the Cosmetic Professional Evaluation Panel, the coverage effect of blemish and pores and comfort finish were evaluated according to the following standards. The long-lasting effect was evaluated according to the following criteria 4 hours or 8 hours after application. Non-transfer to mask was assessed 4 hours or 8 hours after application of a surgical mask according to the following criteria. A “blemish” is a portion that is darker than the surrounding area due to the deposition of a pigment such as melanin on the surface of the skin or in the skin. In this test, it was assumed that the cosmetic composition applied to the skin in the morning was evaluated for its condition at the evening time.

Criteria for Non-Transfer to Mask
A: Very excellent (e.g. almost no transfer of the cosmetic to mask even after 8 hours)
B: Excellent (e.g. almost no transfer of the cosmetic to mask after 4 hours, but 1 to 2 people have a transfer of the cosmetic to mask after 8 hours.)
C: Slightly good (e.g. There is almost no transfer of the cosmetic to mask after 4 hours, but 3 to 5 people have a transfer of the cosmetic to mask after 8 hours.)
D: Slightly bad (e.g. 1 to 2 people have a transfer of the cosmetic to mask after 4 hours)
E: Inferior (e.g. 3 to 5 people have a transfer of the cosmetic to mask after 4 hours.)
F: Very poor (e.g. 6 to 10 people have a transfer of the cosmetic to mask after 4 hours.)

Criteria for Long-Lasting Effect
A: Very excellent (e.g. Makeup does not come off even after 8 hours.)
B: Excellent (e.g. Makeup does not come off after 4 hours, but makeup of 1 to 2 people comes off after 8 hours.)
C: Slightly good (e.g. Makeup does not come off after 4 hours, but makeup of 3 to 5 people comes off after 8 hours.)
D: Slightly bad (e.g. Makeup of 1 to 2 people comes off after 4 hours.)
E: Inferior (e.g. Makeup of 3 to 5 people comes off after 4 hours.)
F: Very poor (e.g. Makeup of 6 to 10 people comes off after 4 hours.)

Criteria for Coverage Effect
A: Very excellent (e.g. Blemish and pores are completely concealed.)
B: Excellent (e.g. No blemish is visible, but pores can be observed for 1 to 2 people.)
C: Slightly good (e.g. No blemish is visible, but pores can be observed for 3 to 5 people.)
D: Slightly bad (e.g. Blemish and pores can be observed for 1 to 2 people.)
E: Inferior (e.g. Blemish and pores can be observed for 3 to 5 people.)
F: Very poor (e.g. Blemish and pores can be observed for 6 to 10 people.)

Criteria for Comfort Finish
A: Very excellent (e.g., No sense of stagnation and no resistance at all to changes in facial expression)
B: Excellent (e.g., Slight sense of stagnation and no resistance to changes in facial expression)
C: Slightly good (e.g. Sense of stagnation but no resistance to changes in facial expression)
D: Slightly bad (e.g. Sense of stagnation and resistance to changes in facial expression)
E: Inferior (e.g. Strong sense of stagnation and resistance to changes in facial expression)
F: Very poor (e.g. Strong sense of stagnation and strong resistance to changes in facial expression, in addition, skin irritation by drying)

[Stability Assessment]

The water-in-oil emulsion cosmetic compositions of Examples and Comparative Examples were placed in a transparent container, sealed with a lid, and stored at 50° C. for 1 month. As a control, a water-in-oil emulsion cosmetic composition stored at room temperature for 1 month was used. The appearance of the water-in-oil emulsion cosmetic composition after storage was visually observed. When any of the following caking and aggregation of the powder was observed in comparison with the control, it was judged as “B: Not good”, and when there was no difference from the control, it was judged as “A: Good”.

Caking: Even if 10 times of shaking by hand, the settled pigment remains solid.

Aggregation of powder: Even if 10 times of shaking by hand, the pigment is unevenly dispersed and creates uneven in color.

The above results are summarized in Tables 4 to 6 below.

TABLE 4

| | Example1 | Example2 | Example3 | Example4 | Example5 | Example6 | Example7 | Example8 | Example9 |
|---|---|---|---|---|---|---|---|---|---|
| Amount of film former | 6 | 2.5 | 3 | 4 | 8 | 12 | 6 | 6 | 8 |
| Amount of low HLB | 2 | 2 | 2 | 2 | 2 | 2 | 1.5 | 4.5 | 1.5 |
| silicone surfactant | | | | | | | | | |
| Amount of non- | 0.24 | 0.24 | 0.24 | 0.24 | 0.24 | 0.24 | 0.24 | 0.24 | 4.74 |
| volatile oil | | | | | | | | | |
| Ratio of film | 2.68 | 1.12 | 1.34 | 1.79 | 3.57 | 5.36 | 3.45 | 1.27 | 1.28 |
| former/(silicone | | | | | | | | | |
| surfactant + | | | | | | | | | |
| non-valatile | | | | | | | | | |
| oils) | | | | | | | | | |
| Amount of polyol | 2 | 2 | 2 | 2 | 2 | 2 | 2 | 2 | 2 |
| Amount of | 42.96 | 46.46 | 45.96 | 44.96 | 40.96 | 36.96 | 43.46 | 40.46 | 36.96 |
| volatile oil | | | | | | | | | |
| Non-transfer effect | A | C | B | A | A | A | A | B | B |
| Long lasting effect | A | C | B | A | A | A | A | A | A |
| High coverage effect | A | C | C | B | A | A | A | A | A |
| Comfort finish | A | A | A | A | B | C | A | B | A |
| Stability | A | A | A | A | A | A | A | A | A |

TABLE 5

| | Exam- | Exam- | Exam- | Exam- | Exam- | Exam- | Exam- | Exam- | Exam- | Exam- |
|---|---|---|---|---|---|---|---|---|---|---|
| | ple10 | ple11 | ple12 | ple13 | ple14 | ple15 | ple16 | ple17 | ple18 | ple19 |
| Amount of | 8 | 9 | 11 | 6 | 6 | 6 | 6 | 6 | 6 | 6 |
| film former | | | | | | | | | | |
| Amount of low | 1.5 | 1 | 1 | 2 | 2 | 2 | 2 | 2 | 2 | 2 |
| HLB silicone | | | | | | | | | | |
| surfactant | | | | | | | | | | |
| Amount of non- | 6.74 | 0.24 | 0.24 | 0.24 | 0.24 | 0.24 | 0.24 | 0.24 | 0.24 | 0.24 |
| volatile oil | | | | | | | | | | |
| Ratio of film | 0.97 | 7.26 | 8.87 | 2.68 | 2.68 | 2.68 | 2.68 | 2.68 | 2.68 | 2.68 |
| former/(silicone | | | | | | | | | | |
| surfactant + | | | | | | | | | | |
| non-volatile | | | | | | | | | | |
| oils) | | | | | | | | | | |
| Amount of polyol | 2 | 2 | 2 | 2 | 2 | 2 | 2 | 2 | 2 | 2 |
| Amount of | 34.96 | 40.96 | 38.96 | 42.96 | 42.96 | 42.96 | 42.96 | 42.96 | 42.96 | 42.96 |
| volatile oil | | | | | | | | | | |
| Non-transfer | C | A | A | A | B | A | B | B | C | C |
| effect | | | | | | | | | | |
| Long lasting | C | A | A | A | A | A | A | A | C | B |
| effect | | | | | | | | | | |
| High coverage | C | A | A | A | A | A | A | A | B | B |
| effect | | | | | | | | | | |
| Comfort finish | B | B | C | B | C | B | C | B | A | A |
| Stability | A | A | A | A | A | A | A | A | A | A |

TABLE 6

| | Comp. | Comp. | Comp. | Comp. | Comp. | Comp. |
|---|---|---|---|---|---|---|
| | Example | Example | Example | Example | Example | Example |
| | 1 | 2 | 3 | 4 | 5 | 6 |
| Amount of film former | 0 | 6 | 6 | 7 | 12 | 6 |
| Amount of low HLB | 2 | 8 | 2 | 3 | 1 | 2 |
| silicone surfactant | | | | | | |
| Amount of non-volatile oil | 0.24 | 0.24 | 10.24 | 4.74 | 0.24 | 0.24 |
| Ratio of film | 0.00 | 0.73 | 0.49 | 0.90 | 9.68 | 2.68 |
| former/(silicone | | | | | | |
| surfactant + | | | | | | |
| non-valatile | | | | | | |
| oils) | | | | | | |
| Amount of polyol | 2 | 2 | 2 | 2 | 2 | 2 |
| Amount of volatile oil | 48.96 | 36.96 | 32.96 | 40.96 | 37.96 | 42.96 |
| Non-transfer effect | F | F | F | D | A | D |
| Long lasting effect | F | E | E | D | A | E |
| High coverage effect | E | C | D | C | A | C |
| Comfort finish | C | F | C | A | F | E |
| Stability | B | A | A | A | B | A |

1. A water-in-oil emulsified cosmetic composition comprising
a hydrophobized pigment (A),
a silicon film former (B),
a silicone-based surfactant having an HLB value of 2 to 8 (C),
and optionally a non-volatile oil (D),
wherein component (A) is at least one kind of an inorganic pigment except for calcium phosphate, which is treated with a hydrophobizing agent except for a bisbenzoyl compound,
wherein the ratio of the mass of component (B) to the total mass of component (C) and component (D) is 1.0 to 9.0, and
wherein the composition contains no fluoroether compound.

2. The water-in-oil emulsion cosmetic composition according to claim 1, wherein the hydrophobizing agent is at least one compound selected from the group consisting of a silicone compound, a silane compound, an amino acid derivative, a sugar derivative, an organic titanate, a phospholipid, a metal soap, a fatty acid, an oil and an organic polymer.

3. The water-in-oil emulsion cosmetic composition according to claim 1, wherein the component (B) is at least one compound selected from the group consisting of trialkylsiloxysilicate, polyalkylsilsesquioxane, (meth) acrylate silicone and carbosiloxane dendrimer.

4. The water-in-oil emulsion cosmetic composition according to claim 1, further comprising at least one compound selected from the group consisting of a polyol, a spherical powder, an organically modified clay mineral and a volatile oil.

5. The water-in-oil emulsion cosmetic composition according to claim 4, wherein the content of the polyol is less than 6% by mass based on the total amount of the cosmetic composition.

6. The water-in-oil emulsion cosmetic composition according to claim 4, wherein the content of the volatile oil is 25 to 55% by mass based on the total amount of the cosmetic composition.

7. The water-in-oil emulsion cosmetic composition according to claim 1, wherein the viscosity of the composition at 25° C. is 1000 mPa·s or less.

8. The water-in-oil emulsion cosmetic composition according to claim 1, wherein the composition has a two-phase shaking form.

9. A cosmetic process for caring for and/or making-up keratinic materials, comprising the application onto keratinic materials, in particular onto skin, of the water-in-oil emulsified cosmetic composition according to claim 1.

10. A cosmetic process according to claim 9, wherein the water-in-oil emulsified cosmetic composition provides to the keratinic materials on which it is applied, non-transfer effect, long-lasting effect, high coverage effect and comfort finish.
